(19) United States
(12) Patent Application Publication
(10) Pub. No.: US 2002/0177733 A1
(43) Pub. Date: Nov. 28, 2002

(54) Manufacture of cyclic aliphatic acids and esters

(76) Inventor: Ramzi Saleh, Baton Rouge, LA (US)

Correspondence Address: ANDREW B. GRIFFIS, ESQ., EXXONMOBILE CHEMICAL COMPANY, P.O. BOX 2149, BAYTOWN, TX, 77522, US

(21) Appl. No.: 09/819,937

(22) Filed: Mar. 28, 2001

(51) Int. Cl. C07C 51/12

(52) U.S. Cl. 562/497

(57) ABSTRACT

Production of cyclic aliphatic acids and esters are achieved in high yield via carbonylation of a cyclic olefin with carbon monoxide in the presence of catalyst.

### BACKGROUND OF THE INVENTION

1. Field of the Invention

The present invention relates to a process for the manufacture of cyclic aliphatic acids or esters via carbonylation of corresponding cyclic olefins with CO in the presence of a Lewis Bronstead acid with sufficient strength to effect carbonylation. Specifically, the present invention describes a process for the synthesis of cyclopentane carboxylic acid or its esters from cyclopentene (CPTE) containing streams using a borontrihalide type catalyst. The CPTE can be produced on demand as a side stream during the production of cyclopentane (CPTA).

2. Description of Related Art

The present invention provides a novel method for the preparation of cyclic aliphatic acids and olefins. Other references in the art describe production of carboxylic acids from linear olefins using various methods of carbonylation. What is unique about the present invention is the unexpected conversion of a ‘cyclic’ olefin resulting in high yield of a cyclic aliphatic acid or ester from a conventional CPTA process. The examples to follow involve production of cyclopentane carboxylic acid or its esters from a cyclopentene stream via carbonylation with CO in the presence of catalyst. The advantage of the present process invention for the examples, is that it allows the use of low cost feed stock, such as dicylopentadiene, easy separation of the product, and catalyst recycle, and exceptionally high selectivity to the acid. The product is disengaged from its complex with the BF₃.2H₂O, by adding the equivalent mole of water as the starting cyclopentene. This reconstitute the BF₃.2H₂O as a bottom phase that can be recycled to the reactor.

### SUMMARY OF THE INVENTION

The present invention is directed to a method for the production of cyclic aliphatic acids or esters comprising (a) reacting a cyclic olefin with carbon monoxide in the presence of an acid catalyst to produce a cyclic carbonium ion; and (b) reacting the cyclic carbonium ion with water; thereby producing a cyclic aliphatic acid or ester. In one embodiment of the invention, the partial pressure of carbon monoxide in step (a) is in the amount from about 500 psig to about 3000 psig. In another embodiment of the invention, the temperature is within the range of from about 25° C. to about 250° C. In a further embodiment of the invention, the cyclic olefin in step (a) is introduced gradually. Preferably, the molar ratio of acid catalyst to cyclic olefin is about 2:1. Also preferably, the catalyst is selected from the group including borontrihalide, sulfuric acid, WO₃/Al₂O₃, SiO₂/Al₂O₃, HF, H—Y Zeolite, H-Mordenite, ZrO₂/H₂SO₄, Nafion, ZrO₂, Ammonium 12-tugstophosphoric acid; CF₃SO₃H, H₃PW₁₂O₄₀, AlCl₃, HF-NbO₅, HSO₃Cl, SbF₅/SiO₂-Al₂O₃, AlCl₃/CuSO₄, AlCl₃/CuCl₂, H₂S₂O₇, ZrO₂/SO₄⁻², TiO₂/SO₄⁻², FSO₃H, HF-SbF₅, FSO₃H—SO₃, FSO₃H-AsF₅, FSO₃H-TaF₅, FSO₃H-SbF₅ and mixtures thereof. Preferably, the acid catalyst is a borontrihalide. Also preferably, the amount of water added to the reaction mixture after the reaction is complete, to free the product and and recycle catalyst is a molar ratio of water to cyclic olefin is about 1:1.

In an embodiment of the invention, the cyclic olefin is a cyclopentene. In another embodiment of the invention, the cyclopentene is obtained by a process comprising (a) thermally cracking dicyclopentadiene to produce cyclopentadiene; (b) reacting cyclopentadiene with hydrogen gas to produce cyclopentene.

The present invention is also directed to a method for the production of cyclopentane carboxylic acid or cyclopentane ester comprising the steps of (a) thermally cracking dicyclopentadiene to produce cyclopentadiene; (b) reacting cyclopentadiene with hydrogen gas to produce a mixture of cyclopentane and cyclopentene; (c) reacting the cyclopentene with carbon monoxide in the presence of an acid catalyst to produce a cyclic carbonium ion; d. reacting the cyclic carbonium ion with water thereby producing cyclopentane carboxylic acid or cyclopentane ester. Preferably, the molar ratio of acid catalyst to cyclopentene is about 2:1.

Also preferably, the acid catalyst is a borontrihalide. In an embodiment of the invention, the borontrihalide catalyst is regenerated by addition of water in step (d). In another embodiment of the invention, the molar ratio of water to cyclopentene is about 1:1. Preferably, the partial pressure of carbon monoxide in step (a) is in the amount from about 500 psig to about 3000 psig. Also preferably, the temperature is within the range of from about 25 to about 250° C.

In an embodiment of the invention, the cyclopentane is removed from the mixture either before introducing the cyclopentene to carbon monoxide in step (c) or after addition of water to the reaction in step (d).

### DESCRIPTION OF THE FIGURES

FIG. 1 is a schematic diagram of the process for synthesis of cyclopentane carboxylic acid starting from a mixture of cyclopentene and cyclopentane according to the present invention.

FIG. 2 is a schematic diagram of the process for synthesis of cyclopentane carboxylic acid starting from dicyclopentadiene according to the present invention.

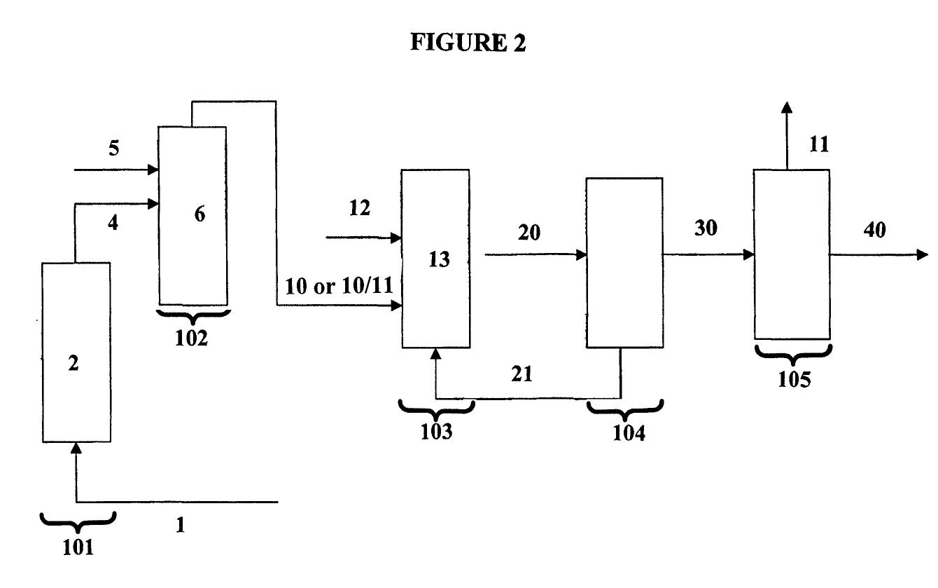

### DETAILED DESCRIPTION OF THE INVENTION

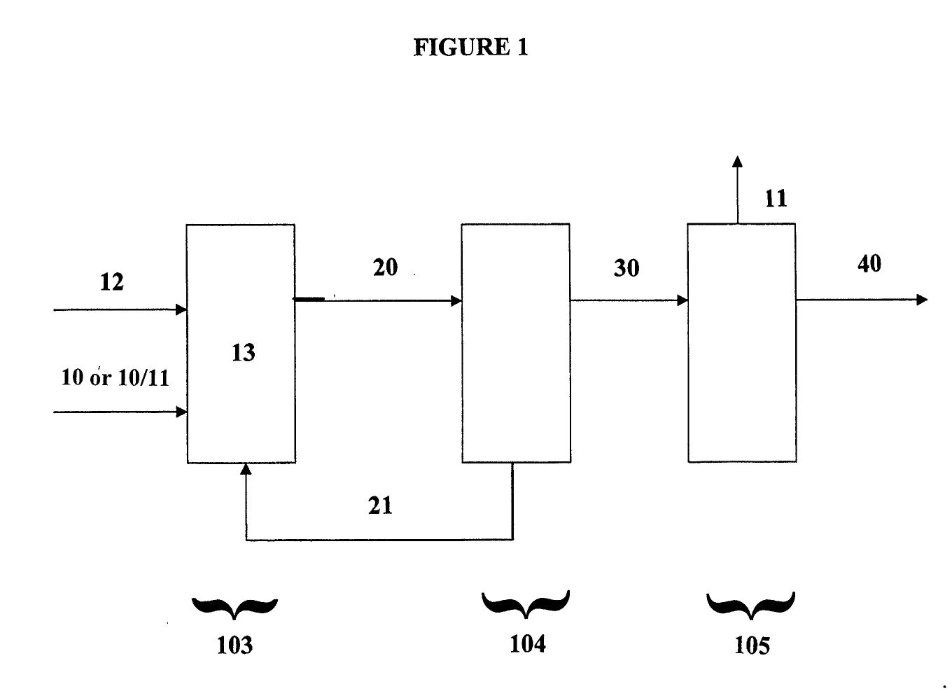

The basic process for synthesis of cyclopentane carboxylic acid or its esters, as shown in FIG. 1, is the carbonylation of cyclopentene 10 with CO 12 in the presence of a catalyst 13, and then adding water 20. The cyclopentene/cyclopentane 10/11 streams undergo carbonylation with CO in the presence of a catalyst such as BF₃.2H₂O,. Only cyclopentene will be converted to cyclopentanecarboxylic acid 40 with using BF₃.2H₂O, leaving behind cyclopentane 11 unconverted. The resulting cyclopentane carboxylic acid and unconverted cyclopentane form a single upper organic phase including the crude acid 30, when quenching the reaction mixture with water 20 to reconstitute the starting BF₃.2H₂O by hydrolysis 104. The reconstituted BF₃.2H₂O 21 is recycled back to facilitate carbonylation 103. The single upper phase is then placed in a distillation tower for finishing 105 whereby the cyclopentane 11 goes overhead and the pure acid remains.

The following equation shows BF₃.2H₂O generating the acid:

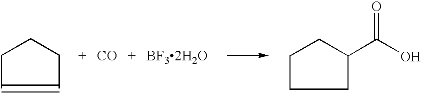

The esters are easily made by reaction of the cyclic acid with an appropriate alcohol in the presence of a suitable esterification catalyst such as p-toluene sulfonic acid, sulfuric acid, or titanium isopropoxide.

A catalyst having a Hammett acidity of <−7 may be used in place of the borontrihalide catalyst shown. Other suitable catalysts include sulfuric acid, trifluoromethanesulfonic acid, ionic solids such as AMBERLYST-15 (ionic exchange resin), WO₃/Al₂O₃, SiO₂/Al₂O₃, HF, H—Y Zeolite, H-Mordenite, ZrO₂/H₂SO₄, Nafion, ZrO₂, Ammonium 12—tugstophosphoric acid; CF₃SO₃H, H₃PW₁₂O₄₀, AlCl₃, HF-NbO₅, HSO₃Cl, SbF₅/SiO₂-Al₂O₃, AlCl₃/CuSO₄, AlCl₃/CuCl₂, H₂S₂O₇, ZrO₂/SO₄⁻², TiO₂/SO₄⁻², FSO₃H, HF-SbF₅, FSO₃H-SO₃, FSO₃H-AsF₅, FSO₃H-TaF₅, FSO₃H-SbF₅ and mixtures thereof.

The following Experiments are presented to illustrate the invention, but the invention is not to be considered as limited thereto.

Experiment

1

800 g of BF₃.2H₂O was charged to a one-liter stirred autoclave. The autoclave was pressurized with CO at 1250 psig, then heated to 55° C. The autoclave was maintained at these conditions, with continuous stirring, for one hour. 250 cc of a 50/50 (w/w) mixture of cyclopentene/cyclopentane was slowly added to the autoclave over a period of 2.75 hours. After adding the feed, the autoclave was kept for an additional three hours at same conditions. The autoclave was cooled to room temperature, vented, then drained into a vessel containing 340 g of water. Two distinct phases developed: an upper phase containing the reaction products and a lower phase containing the catalyst. GC analysis of the upper phase showed that the majority of the cyclopentene was converted to cyclopentanecarboxylic acid, in addition to some higher oligomers of the starting feed. The cyclopentane in the starting feed was largely intact.

Experiment 2

The experiment is performed similarly to Experiment 1, except that the autoclave temperature was set at 120° C., and the autoclave was immediately cooled after adding the feed. The reaction mixture, upon mixing with water, developed two phases: an upper phase whose GC trace was essentially the same as in Experiment 1, and a lower phase containing the catalyst.

Table 1 shows performance data for four runs carried out in the one-liter autoclave, two runs at 55° C. and two runs at 120° C.

TABLE 1
Carbonylation Data of Cyclopentene over BF₃.2H₂O

| | Soak time = 3 hours* | | Soak time = 0 hours** | |
|---|---|---|---|---|
| Temperature, | % Selectivity | % Selectivity | % Selectivity | % Selectivity |
| ° C. | Acid | Heavies | Acid | Heavies |
| 55 | 46 | 54 | 38 | 62 |
| 120 | 92 | 8 | 88 | 12 |

*Reactor kept at conditions for 3 hours then quenched.
**Reactor quenched immediately after the feed was charged to autoclave.

The reaction conditions include a carbon monoxide partial pressure of 1250 psig, a comparison in reaction temperature of 55° C. and 120° C., and soak time of three hours versus no soak time. Cyclopentane/Cyclopentane mixture was charged to reactor over 2.75 hours. In all runs, the cyclopentene was completely converted and the cyclopentane was unaltered. The three-hour soak time or reaction time kept the reaction at conditions for 3 hours before quenching. As shown, the difference in soak time significantly affected the % yield of acid. The highest yield of acid occurred at 120° C. with a soak time of 3 hours. While this Table indicates that an increase in soak time and temperature provide higher yield of acid, other factors not in consideration for the Table may further improve the acid yield. These factors include rate of addition of feed, catalyst to cyclopentene ratio, minimization of by-products. Preventing the heavies or heavy by-products from forming is a key factor in increasing acid yield.

Avoidance of oligomerization is an objective, which can be controlled by four major factors thereby contributing to the high percent yield of cyclopentane carboxylic acid or ester. The four factors are: (1) addition of CO; (2) use of excess amounts of catalyst; (3) low amounts of cyclopentene; and (4) slow addition of cyclopentene. Addition of carbon monoxide at partial pressure is necessary for preventing oligomerization because the carbon monoxide competes with other cyclopentenes for the cyclopentene that has combined with catalyst to form a carbonium ion. Use of high amounts of catalyst is critical for maximizing the reaction between cyclopentene and carbon monoxide. The more catalyst that is added to the reaction mixture, the more cyclopentenes are combined with the catalyst to form carbonium ions. An excess of catalyst is preferable, and a molar ratio of about 2:1, catalyst to cyclopentene, is more preferable for driving the reaction. Two other factors of importance are the addition of low amounts of cyclopentene or slow addition of cyclopentene, both of which help prevent large amounts of cyclopentenes from being in close proximity to each other and interacting for oligomerization.

Comparatively, cyclopentene is more reactive towards oligomerization than a linear olefin. Therefore, preventing cyclopentene from oligomerizing requires careful study of the above factors in order to tailor a commercially viable process for high yield of the cyclic carboxylic acid or ester. The present invention is a novel process for cyclopentene carbonylation, which balances the discussed factors for preventing oligomerization, and accomplishes the goal of providing a commercially viable process for high yield of carboxylic acid or ester. Additional considerations may also be incorporated to make the process more commercially savvy.

As described in Experiment 1, a mixture of cyclopentene/cyclopentane is added to the autoclave. The cyclopentane is not necessarily required to drive the reaction and thus can be taken out by flashing off. In fact, one advantage for removing the cyclopentane is to increase throughput of cyclopentene and consequently increasing reaction efficiency. However, the presence of cyclopentane in the reaction mixture also has an advantage. Cyclopentane may serve to minimize oligomerization by preventing cyclopentene molecules from physically interacting with each other. Cyclopentane can operate as a control for these competing advantages. What is most important about cyclopentane in the reaction, is that it is non-reactive and can be present in the reaction mixture because pure cyclopentene is not easily attainable as a starting material.

While cyclopentene is not a widely available starting material, it can be produced from dicyclopentadiene 1, which is both inexpensive and accessible. Dicyclopentadiene is fed into a catalytic distillation column 2 via a conduit along with up to 20% of a diluent solvent, and preferably enters catalytic distillation column at a location below the catalytic zone. The dicyclopentadiene is cracked to cyclopentadiene 4 within the bottom cracking zone or reboiler of catalytic distillation column. Hydrogen, preferably in the form of H₂ gas 5, is also fed into catalytic distillation column 6 via conduit. It is preferred that the hydrogen gas also be fed into catalytic distillation column at a location below the catalytic zone. Vapor phase cyclopentadiene, produced by cracking 101 dicyclopentadiene, is then hydrogenated 102 in the presence of the hydrogen gas and in the presence of a hydrogenation catalyst in catalytic zone to form cyclopentane. The vapor phase cyclopentadiene is diluted by saturated liquid flowing down the column and hydrogenated as it is produced in catalytic distillation column, thus suppressing a cyclopentadiene polymerization reaction, which would otherwise occur. The resulting cyclopentane is selectively distilled as a vapor phase away from catalytic zone as it is formed. The cyclopentane vapor phase can be taken as a side stream from a top distillation portion of column, or can be taken overhead to condenser via conduit for further concentration before being released to conduit. Unreacted treatgas, including hydrogen and any other treatgas impurities (e.g. methane or ethane) is removed overhhead from condenser as an off gas via conduit. Heavy by-products are taken as bottoms via conduit, and can be recycled to the cracking zone via conduit and reboiler. Alternatively, these heavies can be purged from the system via conduit. Production of cyclopentene is the same process as above while controlling the hydrogenation.

TABLE 2
Operating Parameters for Production of Cyclopentane
via Catalytic Distillation

| | |
|---|---|
| Pressure (psig) | 8 |
| Temperature (° F.) | 175 (isothermal) |
| Catalyst | Massive nickel hydrogenation |
| | catalyst |
| WHSV (weight hourly space | 0.08 cyclopentane |
| velocity-lb/hour/lb of catalyst) | |
| Treatgas Ratio (hydrogen to feed | 150% of stochiometric |
| ratio) | |
| Reflux ratio (R/D) | 1.0 |
| Conversion | >99% |

Another marketable aspect of the subject process is its ability to regenerate catalyst (trihalide catalysts). As described in the Experiments, the catalyst, BF₃.2H₂O, combines with cyclopentene. Addition of water in an amount that is at least a 1:1 molar ratio, catalyst to cyclopentene, is included to regenerate the water complexed with the catalyst.

Overall, the subject process serves to upgrade by conversion, a relatively uncommercial material to more versatile industrial components. Cyclopentane carboxylic acid has great utility over olefins such as its use in producing amides, alcohols, esters and other intermediates for pharmaceutical and agricultural and specialty chemicals. Esters can be used as oxygenated solvents, polyol esters, lubricants, fragrances, solvents for coatings such as paints, starting materials for synthesis of amides, alcohols, ethers, and ketones. The upgrade to a more valuable material using the subject process is paramount from a business prospective, especially since the process can be made with inexpensive starting materials and can regenerate catalyst.

What is claimed is:

1. A method for the production of cyclic aliphatic acids or esters comprising:
a. reacting a cyclic olefin with carbon monoxide in the presence of an acid catalyst to produce a cyclic carbonium ion; and
b. reacting said cyclic carbonium ion with water;
thereby producing a cyclic aliphatic acid or ester.

2. The method of claim 1, wherein the partial pressure of carbon monoxide in step (a) is in the amount from about 500 to about 3000 psig.

3. The method of claim 1, wherein cyclic olefin in step (a) is introduced gradually.

4. The method of claim 1, wherein the molar ratio of acid catalyst to cyclic olefin is about 2:1.

5. The method of claim 1, wherein said acid catalyst is selected from the group consisting of: borontrihalide, sulfuric acid, WO₃/Al₂O₃, SiO₂/Al₂O₃, HF, H—Y Zeolite, H-Mordenite, ZrO₂/H₂SO₄, Nafion, ZrO₂, Ammonium 12- tugstophosphoric acid; CF₃SO₃H, H₃PW₁₂O₄₀, AlCl₃, HF-NbO₅, HSO₃ Cl, SbF₅/SiO₂-Al₂O₃, AlCl₃/CuSO₄, AlCl₃/CUCl₂, H₂S₂O₇, ZrO₂/SO₄⁻², TiO₂/SO₄⁻². FSO₃H, HF-SbF₅, FSO₃H-SO₃, FSO₃H-AsF₅, FSO₃H-TaF₅, FSO₃H-SbF₅ and mixtures thereof.

6. The method of claim 5, wherein said acid catalyst is a borontrihalide.

7. The method of claim 1, wherein the molar ratio of water to cyclic olefin is about 1:1.

8. The method of claim 1, wherein the cyclic olefin is a cyclopentene, or methylcyclopentene.

9. The method of claim 8, wherein the cyclopentene is obtained by a process comprising:
a. thermally cracking dicyclopentadiene to produce cyclopentadiene;
b. reacting cyclopentadiene with hydrogen gas to produce cyclopentene.

10. The method of claim 1, the temperature is within the range of from about 25° C. to about 250° C.

11. A method for the production of cyclopentane carboxylic acid or cyclopentane ester comprising the steps of:
a. thermally cracking dicyclopentadiene to produce cyclopentadiene;
b. reacting cyclopentadiene with hydrogen gas to produce a mixture of cyclopentane and cyclopentene;
c. reacting said cyclopentene with carbon monoxide in the presence of an acid catalyst to produce a cyclic carbonium ion;
d. reacting said cyclic carbonium ion with water thereby producing cyclopentane carboxylic acid or cyclopentane ester.

12. The method of claim 11, wherein the molar ratio of acid catalyst to cyclopentene is about 2:1.

13. The method of claim 11 , wherein said acid catalyst is selected from the group consisting of: borontrihalide, sulfuric acid, WO₃/Al₂O₃, SiO₂/Al₂O₃, HF, H—Y Zeolite, H-Mordenite, ZrO₂/H₂SO₄, Nafion, ZrO₂, Ammonium 12- tugstophosphoric acid; CF₃SO₃H, H₃PW₁₂O₄₀, AlCl₃, HF-NbO₅, HSO₃Cl, SbF₅/SiO₂-Al₂O₃, AlCl₃/CuSO₄, AlCl₃/CuCl₂, H₂S₂O₇, ZrO₂/SO₄⁻², TiO₂/SO₄⁻², FSO₃H, HF-SbF₅, FSO₃H-SO₃, FSO₃H-AsF₅, FSO₃H-TaF₅, FSO₃H-SbF₅ and mixtures thereof.

14. The method of claim 13, wherein said acid catalyst is a borontrihalide.

15. The method of claim 14, wherein said borontrihalide catalyst is regenerated by addition of water in step (d).

16. The method of claim 11, wherein the molar ratio of water to cyclopentene is about 1:1.

17. The method of claim 11, wherein the partial pressure of carbon monoxide in step (a) is in the amount from about 500 psig to about 3000 psig.

18. The method of claim 11, wherein the cyclopentane is removed from the mixture either before introducing the cyclopentene to carbon monoxide in step (c) or after addition of water to the reaction in step (d).
